(19) United States
(12) Patent Application Publication
(10) Pub. No.: US 2025/0367127 A1
(43) Pub. Date: Dec. 4, 2025

(54) MICROCAPSULE LOADED WITH AN ACTIVE SUBSTANCE AND COMPRISING A MICROMETRIC OPENING

(71) Applicants: CENTRE NATIONAL DE LA RECHERCHE SCIENTIFIQUE, PARIS (FR); UNIVERSITÉ DE LORRAINE, NANCY (FR); INSTITUT GUSTAVE ROUSSY, VILLEJUIF (FR); UNIVERSITÉ PARIS-SACLAY, GIF-SUR-YVETTE (FR)

(72) Inventors: Stefan MC MURTRY, LAXOU (FR); Omar EL MAZRIA, SEICHAMPS (FR); Luis MIR, VERRIÈRES LE BUISSON (FR); Franck ANDRE, VILLABÉ (FR)

(21) Appl. No.: 18/875,099

(22) PCT Filed: Jun. 14, 2023
(86) PCT No.: PCT/EP2023/065980

§ 371 Date: Dec. 13, 2024

(30) Foreign Application Priority Data
Jun. 15, 2022 (FR) FR2205830

(51) Int. Cl. A61K 9/50; A61K 31/704

(52) U.S. Cl. CPC A61K 9/5031; A61K 9/5089; A61K 31/704

(57) ABSTRACT

The present disclosure relates to a bio-assimilable polymer microcapsule loaded with at least one active substance and comprising at least one micrometric opening. The disclosure also relates to a method for obtaining said capsule, and to this capsule for its use in the treatment of a pathology, in particular cancer or myopathy.

### CROSS-REFERENCE TO RELATED APPLICATION(S)

This present application is a national stage application of International Patent Application No. PCT/EP2023/065980, filed Jun. 14, 2023, which claims priority to French Patent Application No. 2205830, filed Jun. 15, 2022, the disclosures of which are hereby incorporated by reference in their entireties.

### TECHNICAL FIELD

The present invention relates to a bio-assimilable polymer microcapsule loaded with at least one active substance and comprising at least one micrometric opening.

The invention also relates to a method for obtaining said capsule, and to this capsule for its use in the treatment of a pathology, in particular cancer or myopathy.

### BACKGROUND

The micro- or nano-encapsulation is a technique used to enclose liquids or solids in an envelope (also referred to as a membrane) that isolates them from the outside environment. It is then possible to release their contents into a chosen environment. Their size may vary from a few nanometers to a few hundred microns.

Depending on the molecules encapsulated and the size of the capsules, it is possible, for example, to offer creams containing fragile molecules (vitamins C, beta carotene, etc.), which will only be released when applied, or to vectorize a drug directly to the target cells, thereby reducing quantities and costs and consequently undesirable side effects. In the textile industry, this method allows to encapsulate, for example, a persistent perfume or a cosmetic active ingredient for prolonged action on the skin.

However, the technologies most commonly used are chemical and suffer from a number of limitations. When the encapsulates are in liquid form, it is very complicated to control the size of the drops. It is also difficult to control the thickness of the encapsulant and therefore the content/container ratio. In addition, the capsules obtained in this way typically release the encapsulated molecule or composition by bio-assimilation of the container. And as the thickness of the encapsulant is difficult to control, so is the release time of the encapsulated molecule or composition. In addition, the molecule or composition to be encapsulated is generally in contact with a liquid or solvent during the preparation of the capsules, which may denature it.

When the encapsulation technology is physical, the encapsulated active ingredient is generally released by dissolution of the encapsulant or rupture of the capsule. This release is sudden and therefore non-continuous, making it impossible to administer the active ingredient slowly and in a finely controlled manner.

Microcapsules with a bio-assimilable envelope loaded with at least one active substance have now been developed, said envelope comprising at least one micrometric opening. These capsules allow the release of molecules of interest, for example cytotoxic molecules, over a period of up to several weeks or months as required, followed by gradual disintegration before finally being metabolized. The localized implantation is easy because of the size of the capsules. The treatment is thus targeted, reducing the quantity of the active product required and therefore the cost of the treatment as well as the potential side effects. The body of the capsules is metabolized, so the implantation may be repeated.

### SUMMARY

It is also possible to encapsulate fragile molecules and biological species in this way. Compared with chemical encapsulation, the microcapsules of the invention also allow to considerably increase the content/container ratio.

In addition, the method for obtaining these microcapsules, by the “physical route”, is particularly advantageous because it is based on an approach that consists of manufacturing the encapsulant first and then filling it. One of the advantages of this technique is that the capsules may always be the same size and the encapsulated product never comes into contact with any harmful chemistry. This technology allows to isolate and protect active ingredients in specific quantities until they are slowly released into the tissues.

Thus, according to a first aspect, the invention concerns a microcapsule comprising an outer envelope made of or comprising a bio-assimilable polymer, and loaded at its core with at least one active substance, said outer envelope comprising at least one micrometric opening.

According to a particular embodiment, the size (t_{A}) of the microcapsule ranges from 80 to 2000 μm, in particular from 80 to 800 μm, in particular from 200 to 800 μm, more particularly from 500 to 800 μm.

According to a particular embodiment, the size (t_{A}) of the microcapsule is greater than or equal to 80 μm and less than 2000 μm, notably between 80 and 1950 μm, in particular between 80 and 1900 μm, more particularly between 80 and 1800, 1700, 1600, 1500, 1400, 1300, 1200, 1100, 1000 or 900 μm.

In a particular embodiment, the size (t_{A}) of the microcapsule ranges from 100 nm to 2000 μm, for example greater than 100 nm and less than 2000 μm, in particular from 200, 300, 400, 500, 600, 700, 800 or 900 nm to 2000 μm.

In a particular embodiment, the size (t_{A}) of the microcapsule ranges from 100 nm to 1950 μm, in particular from 200, 300, 400, 500, 600, 700, 800 or 900 nm to 1950 μm.

In a particular embodiment, the size (t_{A}) of the microcapsule ranges from 100 nm to 1500 μm, in particular from 200, 300, 400, 500, 600, 700, 800 or 900 nm to 1500 μm.

In a particular embodiment, the size (t_{A}) of the microcapsule ranges from 100 nm to 800 μm, in particular from 200, 300, 400, 500, 600, 700, 800 or 900 nm to 800 μm.

In a particular embodiment, the size (t_{A}) of the microcapsule is between 1 and 2000 μm, for example greater than 1 and less than 2000 μm, in particular between 5, 10, 20, 30, 40, 50, 60 or 70 and 2000 μm.

In a particular embodiment, the size (t_{A}) of the microcapsule ranges from 1 to 2000 μm, in particular from 5, 10, 20, 30, 40, 50, 60 or 70 to 1950 μm.

In a particular embodiment, the size (t_{A}) of the microcapsule ranges from 1 to 2000 μm, in particular from 5, 10, 20, 30, 40, 50, 60 or 70 to 1500 μm.

In a particular embodiment, the size (t_{A}) of the microcapsule ranges from 1 to 2000 μm, in particular from 5, 10, 20, 30, 40, 50, 60 or 70 to 800 μm. By “size (t_{A}) of the microcapsule” is meant in particular, unless otherwise stated, the largest dimension of the microcapsule. One such reference may relate to microcapsules having at least partially a cylindrical shape, wherein the size (t_{A}) corresponds in particular to the largest dimension of the base of said cylinder.

According to a particular embodiment, the at least one micrometric opening has a size (t_{B}) of between 0.0125(t_{A}) and 0.4(t_{A}), in particular from 0.1(t_{A}) to 0.4(t_{A}).

According to a particular embodiment, the present invention relates to a microcapsule whose outer envelope comprises one or two micrometric openings, in particular one micrometric opening.

According to a particular embodiment, the outer envelope has a thickness of between 0.001(t_{A}) and 0.2(t_{A}), in particular from 0.01(t_{A}) to 0.15(t_{A}), more particularly about 0.1(t_{A}).

According to a particular embodiment, the present invention concerns a microcapsule having at least partially the shape of a cylinder, for example with a circular, oval, rectangular, square or star-shaped base, in particular a straight circular cylinder or parallelepiped, truncated cylinder, cone, truncated cone, spherical or partially spherical, ellipsoid.

By “at least partially” is meant in particular that the microcapsule has the shape of a cylinder, for example with a circular, oval, rectangular, square or star-shaped base, in particular a right circular cylinder or parallelepiped, truncated cylinder, cone, truncated cone, spherical or partially spherical, ellipsoid; or that the microcapsule may be broken down into an assembly of sub-volumes, at least one of these sub-volumes has the shape of a cylinder, for example with a circular, oval, rectangular, square or star-shaped base, in particular a right circular cylinder or parallelepiped, truncated cylinder, cone, truncated cone, spherical or partially spherical ellipsoid.

According to a particular embodiment, the present invention relates to a microcapsule having at least partially the shape of a cylinder, in particular a straight circular cylinder, the largest dimension of the base (t_{A}) of which is between 80 and 800 μm.

According to a more particular embodiment, the present invention relates to a microcapsule having the shape of a cylinder, in particular a straight circular cylinder, the largest dimension (t_{A}) of the base of which is between 250 and 800 μm, in particular between 500 and 800 μm, the outer envelope of which comprises at least one micrometric, in particular circular, opening, the diameter t_{B} of which is between 100 and 300 μm, in particular between 150 and 250 μm, with (t_{B}) being between 0.1(t_{A}) and 0.4(t_{A}).

According to another more specific embodiment, the present invention relates to a microcapsule having the shape of a cylinder, and the outer envelope of which comprises at least one micrometric opening, in particular one micrometric opening, in particular on one of the bases of the cylinder or on the cylindrical surface, or two micrometric openings, in particular on each of the bases of the cylinder, or on the cylindrical surface, the two micrometric openings being for example opposite.

According to a particular embodiment, the present invention relates to a microcapsule having at least partially a cylinder shape, wherein the ratio R of its height (h) to the largest dimension of the base (t_{A}) is between 0.5 to 2, in particular from 0.5 to 1.5, more particularly from 0.5 to 1.

Such a ratio R is likely to allow the microcapsules of the invention to be easily inserted, in particular in vivo, more particularly at the level of a tumor, for example at a plurality of locations, around and/or in the tumor, homogeneously and/or in different directions.

According to a particular embodiment, wherein the bio-assimilable polymer is selected from the group consisting of poly(lactic acid) (PLA), in particular poly(L-lactic acid) (PLLA) or poly(DL-lactic acid) (PDLLA), poly(glycolic acid) (PGA), poly(ε-caprolactone) (PCL), poly(lactic-co-glycolic acid) (PLGA), poly(ortho ester), polyphosphoester, polyphosphazene, polyanhydride, polyamide, polyester-amide, poly(sebacic acid), polyposphazene, poly(dioxanone), polyurethane, polycarbonate, in particular poly(trimethylene carbonate) (PTMC), poly(propylene carbonate) (PPC), copolyester carbonate (PEC), poly(butylene succinate) (PBS), poly(p-dioxanone) (PPDO), poly(methyl methacrylate) (PMMA), polyhydroxyalkanoate (PHA), polybutylene succinate co-adipate (PBSA), polybutylene adipate coterephthalate (PBAT), polymethylene adipate coterephthalate, aliphatic-aromatic copolyester, poly-3-hydroxybutyrate (PHB), poly(hydroxybutyrate-hydroxyvalerate) (PHB/HV), cellulose acetate phthalate, collagen, gelatin, chitosan, chitin, alginate, carrageenan, gums, in particular shellac, guar gum, gum arabic, or tragacanth, poly(amino acid), in particular poly (aspartic acid), and copolymers comprising them.

In a more specific embodiment, the bio-assimilable polymer is chosen from polylactic acids (PLA), polyglycolic acids (PGA) and polycaprolactones (PCL), and copolymers comprising them.

According to a particular embodiment, the active principle (or active substance) is in solid or liquid form.

In a more specific embodiment, the active ingredient (or active substance) is in powder, gel or paste form.

According to an even more specific embodiment, the active ingredient (or active substance) is in powder form.

According to a particular embodiment, the active substance is not dissolved in a solvent, for example DMSO (dimethyl sulphoxide). According to a particular embodiment, the active substance has not been dissolved, prior to encapsulation, in a solvent, for example DMSO (dimethyl sulphoxide).

According to a particular embodiment, the active substance has not been obtained, before or during encapsulation, from a solution containing said active substance, for example by evaporation (in particular under reduced pressure and/or heating), or freeze-drying of said solution.

In a particular embodiment, the active principle (or active substance) is an enzyme or a drug.

In a particular embodiment, the invention concerns a microcapsule, loaded exclusively at its core with at least one active substance.

By “loaded exclusively at its core with at least one active substance” is meant in particular that the core of the microcapsule of the invention consists of the at least one active substance and possibly air and/or an inert atmosphere.

For example, the microcapsule of the invention is devoid, in particular at its core, of a compound that is not the active substance (before encapsulation), for example a polymer, in particular a carrier polymer.

This active substance may be an active molecule or an active pharmaceutical composition. This active pharmaceutical composition is in particular one of the active pharmaceutical compositions that are or have been on the market.

According to a particular embodiment, the invention concerns a microcapsule, the loading of which is devoid of any compound not present in the active substance prior to encapsulation.

The microcapsules of the invention allow the active substance to be encapsulated without the need to first dissolve the active substance. Within the encapsulation framework of the invention, the active substance remains pure, unmodified and therefore undegraded. The encapsulation according to the invention may be carried out at ambient temperature, without dilution or physical or chemical transformation (for example at high or low temperature) of the active substance.

According to a particular embodiment, the invention relates to a microcapsule, which is capable of releasing said at least one active substance into the body of a patient immediately and over a period of one or more months, for example over a period of approximately 60 days.

According to another aspect, the present invention also relates to a plurality of microcapsules, which are as defined above.

In a particular embodiment, the plurality of microcapsules is uniform in size.

By “uniform in size” is meant in particular that the size (t_{A}) of the microcapsules is within ±20, 10 or 5% of the number average size (t_{A}) of the plurality of microcapsules.

According to another aspect, the present invention also relates to a method for preparing a microcapsule or a plurality of microcapsules as defined above, which comprises the following steps:
(i) Providing a sacrificial mold having at least one cavity;
(ii) Covering the wall or walls of the at least one cavity of said mold with a structural composition comprising a bio-assimilable polymer;
(iii) Drying or annealing the structural composition as obtained at the end of step (ii) in order to obtain a mold, the wall or walls of the at least one cavity of which are covered with the cured bio-assimilable polymer;
(iv) Filling the at least one cavity as obtained at the end of step (iii) with at least one active substance;
(v) Sealing the open cavity as obtained at the end of step (iv) by applying a layer of bio-assimilable polymer to this opening of the mold;
(vi) Removing the mold to obtain at least one microcapsule sealed by said layer of bio-assimilable polymer as obtained at the end of step (v);
(vii) Separating the at least one sealed microcapsule as obtained in step (vi) from the remainder of the bio-assimilable polymer layer;
(viii) Perforating the at least one sealed microcapsule as obtained at the end of step (vii); steps (vii) and (viii) may be reversed.

In a particular embodiment, the sacrificial mold consists of or comprises a water-soluble material, in particular a water-soluble polymer, in particular a polymer chosen from polyvinyl alcohol (PVOH or PVAL), polyvinyl acetate (PVA), polyethylene glycol (PEG), polyvinylpyrrolidone (PVP), polyacrylamide, dextrin, casein, dextran, pullulan and cellulose ethers.

In a particular embodiment, the sacrificial mold has a plurality of cavities, in particular a plurality of identical cavities.

In a particular embodiment, the at least one cavity of the sacrificial mold is formed by the action of a laser, in particular a CO₂ laser, or by molding.

According to a particular embodiment, the structural composition comprising a bio-assimilable polymer of step (ii) is a solution of said polymer in an organic solvent, the organic solvent being, for example, an aromatic solvent.

By “aromatic solvent” is meant in particular benzene, or a solvent consisting of or comprising an aromatic ring, in particular benzene, substituted in particular by alkyl groups, for example C₁-C₃, and/or alkoxy groups, for example C₁-C₃.

According to a more particular embodiment, the concentration of polymer in the organic solvent is between 10 and 200 g/L, in particular from about 20 to about 100 g/L.

According to a particular embodiment, step (ii) is repeated, in particular once.

According to a more particular embodiment, the polymer concentration in the organic solvent is about 100 g/L, step (ii) being repeated, in particular once.

According to another, more particular, embodiment, step (ii) is repeated two or more times, the first covering being made with a structural composition comprising a bio-assimilable polymer at approximately 20 g/L, the other coverings being made with a structural composition comprising a bio-assimilable polymer at approximately 100 g/L.

In a particular embodiment, the covering in step (ii) is produced by spin coating, spraying, dipping, fogging, flexographic printing, screen printing, ink jet printing, gravure printing or by coating using a slot die.

According to a particular embodiment, the drying or annealing of the structural composition in step (iii) is an annealing carried out at a temperature of between 50 and 100° C., in particular about 80° C., and/or for a time of between 5 minutes and 5 hours, in particular about 30 minutes.
a. In a particular embodiment, filling in step (iv) is carried out mechanically.

According to a particular embodiment, the bio-assimilable polymer layer of step (v) is obtained by concentrating a solution of said polymer in an organic solvent, the organic solvent being for example an aromatic solvent.

In a particular embodiment, the concentration is achieved by heating, in particular with stirring, and in particular in a polytetrafluoroethylene container.

According to a particular embodiment, the sealing in step (v) is carried out under pressure, in particular under the pressure of an inflatable membrane, in particular made of rubber. Said pressure is equivalent, for example, to a weight of around 10 kg.

According to a particular embodiment, sealing in step (v) is carried out for a period of 1 to 24 hours, for example 6 to 12 hours, in particular at a temperature of between 15 and 100° C., in particular 20 to 25° C.

According to a particular embodiment, step (v) is preceded by a step of preparing said layer of bio-assimilable polymer by bringing this layer into contact with a solution of said polymer in an organic solvent.

According to a particular embodiment, the sacrificial mold consists of or comprises a water-soluble material, in particular a water-soluble polymer, and removal of the mold from step (vi) is carried out by bringing said mold into contact with water, in particular for a period of from 30 minutes to 12 hours, more particularly for about 4 hours.

According to a particular embodiment, contact with water is made by means of a flow of water, in particular at a flow rate of between 0.1 and 20 L/min, in particular from 1 to 10 L/min, for example approximately 6 L/min, in particular at a temperature of between 20 and 25° C.

According to a particular embodiment, the separation of the at least one sealed microcapsule of step (vii) is carried out using a punch or a coaxial needle.

According to a particular embodiment, the perforation of the at least one sealed microcapsule of step (viii) is carried out mechanically, in particular with the aid of at least one point, with the aid of a laser, or with the aid of ultrasound, preferably mechanically, more preferably with the aid of a point.

According to another aspect, the invention also relates to a microcapsule or a plurality of microcapsules as defined above, for its use in the treatment and/or the prevention of a disease, said disease being in particular a cancer, a myopathy or a dermatological disease.

In a particular embodiment, the disease is cancer. In particular, these include:
tumors that may not be operated on, i.e. tumors that may not be removed from the body with simple surgery,
tumors which are very difficult to access (but which are nevertheless accessible to the microcapsule or microcapsules of the invention, for example by means of a needle through which said microcapsule or microcapsules may be introduced), and/or
tumors that are sensitive, in particular exclusively, to substances that are highly toxic by the systemic route. The treatment of the latter therefore requires a local and preferably long-term application, which may be mediated by the microcapsule or microcapsules of the present invention.

### Definitions

As used in this description, the term “about” refers to a range of values within ±10% of a specific value. For example, the term “about 20” comprises the values of 20±10%, i.e., the values of 18 to 22.

For the purposes of this description, the percentages refer to percentages by mass in relation to the total mass of the formulation, unless otherwise stated.

As understood here, the value ranges in the form of “x-y” or “from x to y” or “between x and y” include the bounds x and y as well as the integers between these bounds. For example, “1-5”, or “from 1 to 5” or “between 1 and 5” refer to the integers 1, 2, 3, 4 and 5. The preferred embodiments include each individual integer in the value range, as well as any sub-combination of those integers. For example, the preferred values for “1-5” may comprise the integers 1, 2, 3, 4, 5, 1-2, 1-3, 1-4, 1-5, 2-3, 2-4, 2-5, etc.

### BRIEF DESCRIPTION OF THE DRAWINGS

FIG. 1 shows the diffusion of fluorescein contained in a microcapsule of the invention within a mouse tumor, the tumor having been cut in half to show the location of the capsule (A) and the diffusion of fluorescein within the tumor (A and B).

FIG. 2 shows fluorescence microscopy through the skin of a capsule as in example 4.1. 1: diffusion of doxorubicin; 2: capsule; 3: tissue/skin.

FIG. 3 shows the fluorescence microscopy observation of a capsule according to example 4.1, after the skin covering the capsule has been incised.

FIG. 4 shows a PLA capsule containing Doxoribicin with a 302 μm diameter perforation.

### DETAILED DESCRIPTION

### EXAMPLES

### Example 1: Preparation of Microcapsules of the Invention

Microcapsules of the invention were obtained as follows:
A sacrificial PVA mold comprising a plurality of cylindrical cavities 800 μm deep and 800 μm in diameter, which have been formed in a 1.2 mm thick layer of PVA by CO₂ laser or molding, has been produced;
Two solutions of PLA in an aromatic solvent, one at 20 g/L and the other at 100 g/L, were prepared by dissolving the PLA in the solvent at 50° C. using ultrasound;
The walls of the cavities in said mold were covered by spin coating the 20 g/L PLA solution, then the 100 g/L solution, with the latter step being repeated;
The covered mold was annealed at 80° C. for 30 minutes;
The cavities were filled mechanically with an active substance in powder form;
A closure layer of PLA was prepared by concentrating a solution of PLA in an aromatic solvent under heating and stirring in a Teflon beaker; said layer is a film obtained on the surface of the beaker;
The surface of this layer is treated with the solution remaining in the beaker after the film has been removed;
the cavities are sealed by plating the PLA layer onto the mold, using an inflated (rubber) membrane, for one night at room temperature;
the PVA mold is then dissolved by a flow of water for 4 hours, the water being at a temperature of between 20 and 25° C.;
the mechanical perforation of the microcapsules thus obtained using a point, viewed by a computer-controlled XYZ camera mounted on a microscope;
the microcapsules obtained are isolated using a punch.

PUR (polyurethane) capsules have been obtained in a similar way.

However, the experimental protocol for PUR may differ in the annealing and drying times of the layers on PVA. In addition, the surface modification of the plated closure film is generally carried out with the initial PUR solution.

### Example 2: Study of the Release of an Active Principle Contained in the Microcapsules of the Invention

The release of encapsulated methylene blue (polyurethane, see example 1) was studied in an aqueous medium. This study showed that the release was stable and continuous over several hours to several weeks, depending on the openings, and was confirmed by UV spectroscopy measurements.

In this way, the thickness of the capsule walls and the size of the perforation may be chosen so that diffusion of the active ingredient occurs over the chosen period and metabolization of the capsule only occurs later.

### Example 3: Further Study of the Release of an Active Principle Contained in the Microcapsules of the Invention

In order to verify the diffusion into tumor tissue, polyurethane capsules 800 μm in diameter, as described in Example 1, containing fluorescein, were implanted into mouse tumors to verify diffusion of the latter into the tumor. Fluorescein is a fluorescent dye that is easily detected by its green fluorescent emission.

The implantation was performed using a trocar or simply by placing the capsule at the end of a needle. One microcapsule per tumor was implanted in a subcutaneous tumor model in immunocompetent mice (LPB fibrosarcomas implanted subcutaneously in C57Bl/6 mice).

Once implanted, the capsule diffused the fluorescein within the tumor for 24 hours.

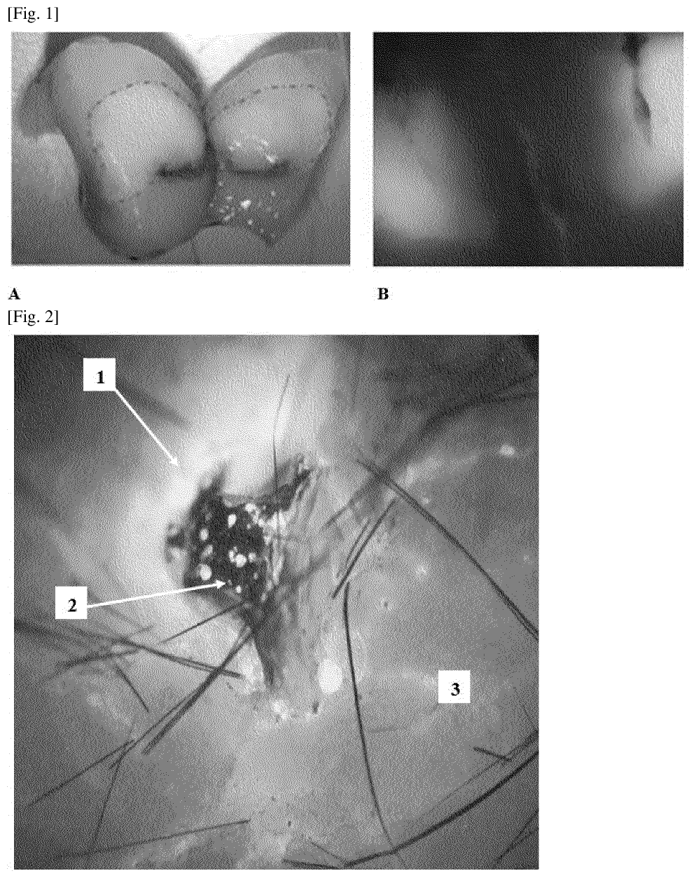

The ablation and the observation of the tumor showed significant and continuous diffusion as described in FIG. 1. However, the microcapsules were not empty after 24 hours: a significant quantity of fluorescein still remained, showing that the release of the content of the microcapsule may extend well beyond 24 hours.

### Example 4: Microcapsules of the Invention for Their Use in Chemotherapy

### 1. Evaluation of the Toxicity of the Microcapsules

A capsule of the invention, obtained according to example 1 and containing doxorubicin, was injected subcutaneously into the right flank of 4 mice (one capsule per mouse).

The mice were then examined at the time of injection and twice a day for two days, to detect any symptoms and/or significant weight loss.

No significant weight loss was observed, nor were there any notable adverse effects.

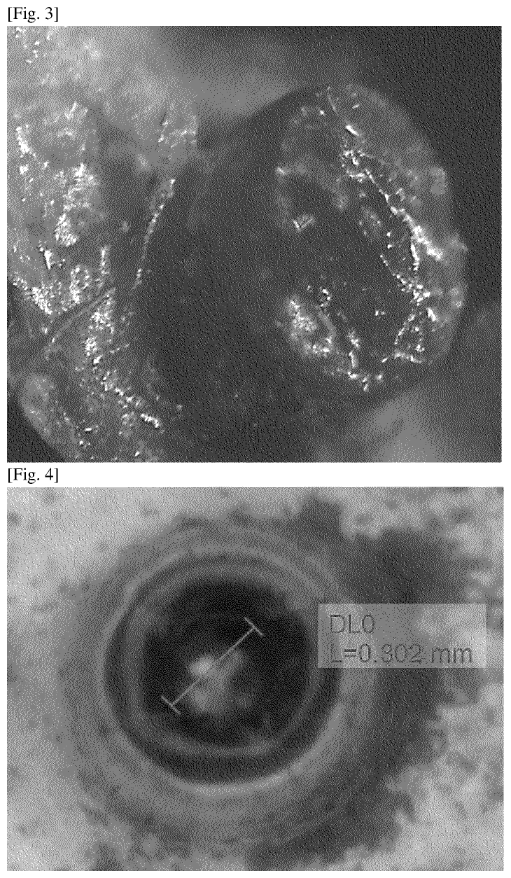

The mice were then autopsied. The capsules were first observed using fluorescence microscopy through the skin. It was shown that all the capsules had diffused into the surrounding tissue, as a luminous halo was observed around the four capsules (FIG. 2). The skin covering the capsules was then incised. The 4 capsules were found in good condition, with a significant quantity of doxorubicin still inside, as observed by fluorescence microscopy (FIG. 3).

### 2. Injection Into a Tumor-Bearing Animal

A capsule of the invention, obtained according to example 1, and containing doxorubicin, was injected into a mouse bearing two tumors, one on the left flank, the other on the right flank (one capsule in each tumor).

The left tumor was cut in half 5 days after injection of the capsules. A diffusion of the doxorubicin is clearly visible, very localized around the capsule, within the tumor. The same applies to the right tumor.

In this way, doxorubicin diffuses into tumors and is thus able to provide an effective, localized chemotherapy treatment using the devices of the present invention.

1. A microcapsule comprising an outer envelope made of or comprising a bio-assimilable polymer and loaded at its core with at least one active substance, said outer envelope comprising at least one micrometric opening.

2. The microcapsule according to claim 1, including:
the size (t_{A}) is greater than or equal to 100 nm and less than 2000 μm; and/or
the at least one micrometric opening has a size (t_{B}) of between 0.0125(t_{A}) and 0.4(t_{A}); and/or
the outer envelope has a thickness of between 0.001(t_{A}) and 0.2(t_{A}).

3. The microcapsule according to claim 1, wherein the bio-assimilable polymer is selected from the group consisting of poly(lactic acid) (PLA), in particular poly(L-lactic acid) (PLLA) or poly(DL-lactic acid) (PDLLA), poly(glycolic acid) (PGA), poly(ε-caprolactone) (PCL), poly(lactic-co-glycolic acid) (PLGA), poly(ortho ester), polyphosphoester, polyphosphazene, polyanhydride, polyamide, polyester-amide, poly(sebacic acid), polyposphazene, poly(dioxanone), polyurethane, polycarbonate, in particular poly(trimethylene carbonate) (PTMC), poly(propylene carbonate) (PPC), copolyester carbonate (PEC), poly(butylene succinate) (PBS), poly(p-dioxanone) (PPDO), poly(methyl methacrylate) (PMMA), polyhydroxyalkanoate (PHA), polybutylene succinate co-adipate (PBSA), polybutylene adipate coterephthalate (PBAT), polymethylene adipate coterephthalate, aliphatic-aromatic copolyester, poly-3-hydroxybutyrate (PHB), poly(hydroxybutyrate-hydroxyvalerate) (PHB/HV), cellulose acetate phthalate, collagen, gelatin, chitosan, chitin, alginate, carrageenan, gums, in particular shellac, guar gum, gum arabic, or tragacanth, poly(amino acid), in particular poly(aspartic acid), and copolymers comprising them.

4. The microcapsule according to claim 1, wherein the active substance:
is not dissolved in a solvent or has not been dissolved in a solvent prior to encapsulation; or
is in solid form, in particular in powder, gel or paste form, or in liquid form, the active substance being in particular in powder form; and/or
is an enzyme or a drug.

5. A method for preparing a microcapsule according to claim 1, which comprises the following steps:
(i) Providing a sacrificial mold having at least one cavity;
(ii) Covering the wall or walls of the at least one cavity of said mold with a structural composition comprising a bio-assimilable polymer;
(iii) Drying or annealing the structural composition as obtained at the end of step (ii) in order to obtain a mold, the wall or walls of the at least one cavity of which are covered with the cured bio-assimilable polymer;
(iv) Filling the at least one cavity as obtained at the end of step (iii) with at least one active substance;
(v) Sealing the open cavity as obtained at the end of step (iv) by applying a layer of bio-assimilable polymer to this opening of the mold;
(vi) Removing the mold to obtain at least one microcapsule sealed by said layer of bio-assimilable polymer as obtained at the end of step (v);
(vii) Separating the at least one sealed microcapsule as obtained in step (vi) from the remainder of the bio-assimilable polymer layer;
(viii) Perforating the at least one sealed microcapsule as obtained at the end of step (vii);
the steps (vii) and (viii) may be reversed.

6. The method according to claim 5, wherein the sacrificial mold consists of or comprises a water-soluble material, in particular a water-soluble polymer, in particular a polymer chosen from polyvinyl alcohol (PVOH or PVAL), polyvinyl acetate (PVA), polyethylene glycol (PEG), polyvinylpyrrolidone (PVP), polyacrylamide, dextrin, casein, dextran, pullulan and cellulose ethers.

7. The method according to claim 5, wherein the structural composition comprising a bio-assimilable polymer of step (ii) is a solution of said polymer in an organic solvent, the organic solvent being for example an aromatic solvent.

8. The method according to claim 5, wherein the bio-assimilable polymer layer of step (v) is obtained by concentrating a solution of said polymer in an organic solvent, the organic solvent being, for example, an aromatic solvent.

9. The method according to claim 5, wherein step (v) is preceded by a step of preparing said layer of bio-assimilable polymer by bringing this layer into contact with a solution of said polymer in an organic solvent.

10. The method according to claim 5, wherein the separation of the at least one sealed microcapsule of step (vii) is carried out using a punch or a coaxial needle.

11. The method according to claim 5, wherein the perforation of the at least one sealed microcapsule of step (viii) is carried out mechanically, in particular with the aid of at least one point, with the aid of a laser, or with the aid of ultrasound, preferably mechanically, more preferably with the aid of a point.

12. The microcapsule according to claim 1, for use in the treatment and/or the prevention of a disease, said disease being in particular a cancer, a myopathy or a dermatological disease.
